(19) United States
(12) Patent Application Publication
(10) Pub. No.: US 2003/0217749 A1
(43) Pub. Date: Nov. 27, 2003

(54) Medical apparatus

(76) Inventor: Silvia Beatriz Dougill, London (GB)

Correspondence Address: Philip H. Von Neida, The BOC Group, Inc., Legal Services - Intellectual Property, 100 Mountain Avenue, Murray Hill, NJ, 07974, US

(21) Appl. No.: 10/440,925

(22) Filed: May 19, 2003

(30) Foreign Application Priority Data
May 23, 2002 (GB) GB 0211906.3

(51) Int. Cl. A61M 15/00; A61M 16/10

(52) U.S. Cl. 128/203.12; 128/203.22

(57) ABSTRACT

An apparatus for delivering a breathable gas together with a nebulised medicament to a patient comprises a gas cylinder 4, a nebuliser 12, a first line 8a extending between the gas cylinder 4 and the nebuliser 12, an outlet from the nebuliser 12 in communication with a mask 14 for use by the patient, and a second line extending between the same or a different gas cylinder 4 and the mask 14 for delivering a breathable gas to the patient. A third line 86 extends between the gas cylinder 4 and the nebuliser 12 for assisting in the delivery of the medicament to the patient.

### FIELD OF THE INVENTION

The present invention relates to medical apparatus for delivering a breathable gas to a patient which also enhances the delivery of nebulised medical drug/formulations to the patient.

For the avoidance of doubt the use of the term “breathable gas” throughout this specification is intended to embrace “breathable gas mixtures” for example oxygen/helium gas mixtures.

### BACKGROUND OF THE INVENTION

Nebulisers are known for delivering medication directly in to the airways and lungs of a patient usually for the treatment of respiratory diseases. Commonly known nebulisers are pneumatically operated using a source of compressed air which atomises a liquid prior to delivery to a patient. One problem associated with known nebulisers is that some at least of the prescribed medication never reaches the lungs of the patient. Of the medication placed in the nebuliser sometimes two thirds remains there at the end of nebulisation. Furthermore, some of the medication released from the nebulisers may be released during expiration by the patient and be lost in to the surrounding air.

U.S. Pat. No. 5,586,551 discloses an oxygen mask having an extended nose region to accommodate openings for both a nebuliser and a main oxygen supply. The nebuliser has a separate oxygen supply tube connected to it.

It is an aim of the present invention to provide an apparatus which not only delivers a breathable gas to a patient but enhances the delivery of nebulised medication to the patient.

### SUMMARY OF THE INVENTION

According to the present invention, an apparatus for delivering a breathable gas together with a nebulised medicament to a patient comprises a source of gas under pressure, a nebuliser, a first line extending between the source and the nebuliser such that gas from the source when passing through the line will operate the nebuliser, an outlet from the nebuliser in communication with a mask for use by the patient; and a second line extending between a source of breathable gas and the mask for delivering a breathable gas to the patient, characterised in that the apparatus additionally comprises a third line extending between the source of gas under pressure and the nebuliser.

The presence of the third line enables delivery of nebulised medication to the patient to be maintained throughout the inspiratory phase of a patient's breathing cycle.

The first line preferably extends to the base of the nebuliser. The third line preferably extends to the upper end of the nebuliser.

In one embodiment, the source of gas under pressure and the source of breathable gas is a gas cylinder containing a helium/oxygen gas mixture.

In an alternative embodiment, the source of gas under pressure is a first cylinder of air or oxygen whilst the source of breathable gas is a second cylinder containing a helium/oxygen gas mixture. Alternatively, the positions of the first and second cylinders may be reversed, i.e. the first cylinder may be of a helium/oxygen gas mixture and the second cylinder of oxygen or air.

A demand valve or a conserver may be located in the second line. Another demand valve or conserver may be located in or upstream of the first line.

This offers the advantage that the nebulised medicament is released from the nebuliser only during inspiration by the patient.

### BRIEF DESCRIPTION OF THE DRAWINGS

Embodiments of the invention will now be described, by way of example, reference being made to the Figures of the accompanying diagrammatic drawings in which:

FIG. 1 is a schematic view of an apparatus for delivering a breathable gas to a patient together with a nebulised medicament according to the present invention; and

FIG. 2 is a schematic view of a modified apparatus similar to the apparatus of FIG. 1.

### DETAILED DESCRIPTION OF THE INVENTION

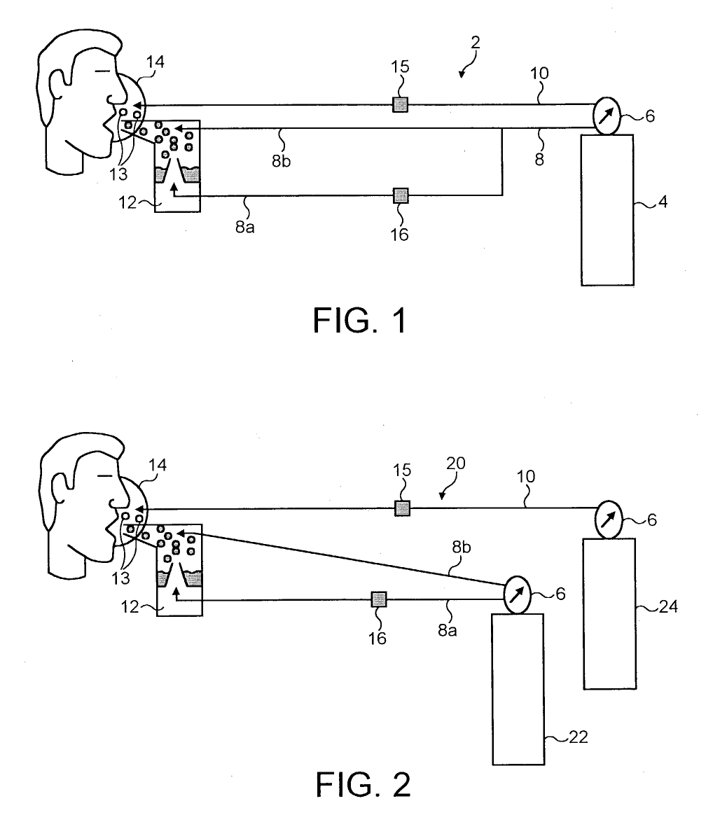

Referring first to FIG. 1, there is shown an apparatus 2 for delivering a breathable gas, for example, a helium-oxygen gas mixture to a patient together with a nebulised medicament. The helium-oxygen mixture preferably contains from 20 to 40% by volume of oxygen and from 60 to 80% by volume of helium. The apparatus 2 includes a source of the gas under pressure in the form of a gas cylinder 4. A gas regulator 6 is fitted to the top (as shown) of the cylinder 4 in a manner known per se and from the regulator 6 extends first and second lines 8, 10 to a nebuliser 12 containing a liquid medicament. The outlet from the nebuliser 12 is in communication with a mask 14 for use by the patient. The mask 14 is formed with one or more apertures 13 for the flow out of the mask 14 of exhaled gas when the patient breathes out. If desired, the apertures 13 may be fitted with flap valves (not shown).

As shown, the first line 8 is branched so that branch 8a extends towards the base of the nebuliser and the branch 8b extends towards the upper end of the nebuliser. As shown, located in the branch 8a is a first demand valve 16.

The second line 10 extends directly from the regulator 6 to the mask 14 and has a second demand valve 15 disposed therein.

In operation, gas under pressure from the cylinder 4 will on inspiration by the patient, pass through line 8 and in to the nebuliser 12. The gas passing through the branch 8a will function to operate the nebuliser 12 by nebulising the liquid medicament which will then pass out from the nebuliser 12 to the mask 14 and hence in to the lungs and airways of the patient. The gas passing through the branch 8b in to the nebuliser 12 helps to draw more of the nebulised medicament in to the lungs/airways of the patient.

The gas passing continuously through the second line 10 to the mask 14 provides a constant supply of breathable gas to the patient during the inspiratory phases of his or her breathing cycle.

The demand valve 16 opens only during inspiration allowing gas to be drawn through the nebuliser 12. On expiration the valve 16 closes. If desired, the demand valve 16 can be omitted or located upstream of the branching of the line 8. Alternatively, the demand valve 16 can be replaced by a conserver.

Referring now to FIG. 2 where like reference numerals denote like parts, the apparatus 20 includes a first source of gas, for example, air or oxygen under pressure in the form of a cylinder 22 and a second source of breathable gas, for example a helium/oxygen gas mixture in the form of a separate cylinder 24.

Gas regulators 6 are fitted to the respective cylinders 22, 24. A first line 8a extends between the regulator 6 of cylinder 22 and the base of the nebuliser 12 and a branch line 8b extends between the regulator 6 and the upper end of the nebuliser 12.

A second line 10 extends directly from the regulator 6 of cylinder 24 to the mask 14 for use by the patient. The mask 14 has apertures 13 in it for the exit of exhaled gas. A demand valve 15 is located in the second line 10 to ensure that gas is delivered to the patient therethrough only during the inspiratory phases of his or her breathing cycle.

As with the apparatus 2 of FIG. 1, a demand valve 16 is located in the first line 8a and the outlet from the nebuliser 12 is in communication with the mask 14.

In operation, gas under pressure from the cylinder 22 will, on inspiration by the patient pass through the line 8a to operate the nebuliser 12 by nebulising liquid medicament contained within the nebuliser which will then pass out from the nebuliser to the mask 14 and hence to the lungs and airways of the patient. The gas from the cylinder 22 passing through the branch 8b in to the nebuliser 12 helps to draw more of the nebulised medicament in to the lungs/airways of the patient.

The breathable gas from the cylinder 24 passes continuously through the second line 10 to the mask 14 to provide a constant supply of breathable gas to the patient during the inspiratory phases of his or her breathing cycle.

In a modification of the apparatuses shown in FIGS. 1 and 2, the mask 14 may be connected to the nebuliser 12 by an adapter (not shown) having a configuration that ensures that the nebuliser is held in a vertical position. In another modification, the demand valves 15 and 16 are omitted.

Advantages of the apparatus 2, 22 over known nebulisers includes:

a) less medicament wastage;

b) improved deposition in lungs; and

c) no electrical compressors required for operating the nebuliser.

1. An apparatus for delivering a breathable gas together with a nebulised medicament to a patient comprising:
a source of gas under pressure;
a nebuliser;
a first line extending between the source and the nebuliser passing through the line will operate the nebuliser;
an outlet from the nebuliser in communication with a mask for use by the patient; and
a second line extending between a source of breathable gas and the mask for delivering a breathable gas to the patient, characterised in that the apparatus additionally comprises a third line extending between the source of gas under pressure and the nebuliser.

2. The apparatus as claimed in claim 1, in which the source of gas under pressure and the source of breathable gas is the same gas cylinder.

3. The apparatus as claimed in claim 2, in which the gas cylinder contains a helium/oxygen gas mixture.

4. The apparatus as claimed in claim 1, in which the source of gas under pressure is a cylinder of air or oxygen and the source of breathable gas is a cylinder containing a helium/oxygen gas mixture, or vice versa.

5. The apparatus as claimed in claim 1, in which a demand valve is located in the second line.

6. The apparatus as claimed in claim 1, in which a conserver is located in the second line.

7. The apparatus as claimed in claim 1, in which the first line extends to the base of the nebuliser.

8. The apparatus as claimed in claim 1, in which the third line extends to the upper end of the nebuliser.
